(19) United States
(12) Patent Application Publication
(10) Pub. No.: US 2023/0172946 A1
(43) Pub. Date: Jun. 8, 2023

(54) In-situ Gel Forming Ophthalmic Formulations Containing Difluprednate

(71) Applicants: IVIEW Therapeutics, Inc., Doylestown, PA (US); IVIEW Therapeutics (Zhuhai) Co., Ltd., Zhuhai (CN)

(72) Inventors: Bo Liang, Plainsboro, NJ (US); Ming Zhang, Yardley, PA (US); Haizhou Peng, Wuhu (CN)

(73) Assignees: IVIEW Therapeutics, Inc., Doylestown, PA (US); IVIEW Therapeutics (Zhuhai) Co., Ltd., Zhuhai (CN)

(21) Appl. No.: 17/051,625

(22) PCT Filed: Aug. 18, 2020
(86) PCT No.: PCT/US2020/046843

§ 371 Date: Oct. 29, 2020

Related U.S. Application Data
(60) Provisional application No. 62/888,534, filed on Aug. 18, 2019.

(51) Int. Cl. A61K 31/567; A61K 47/26; A61K 47/10; A61K 47/44; A61K 9/00; A61K 9/06

(52) U.S. Cl. CPC A61K 31/567; A61K 47/26; A61K 47/10; A61K 47/44; A61K 9/0048; A61K 9/06

(57) ABSTRACT

The present invention provides aqueous in-situ gel ophthalmic formulations, each including water, difluprednate and a biocompatible polysaccharide, wherein a gel is formed in situ at physiological temperature with instant viscosity increase upon instillation of the formulation into an eye. In the formulations, nanocarriers may be formed by a surfactant or solubilizer contained in the formulation, with or encapsulating difluprednate, and the nanocarriers have an average particle size of 10 to 500 nm or 10 to 150 nm or 10- to 50 nm.

### CROSS-REFERENCE TO RELATED APPLICATION

This application claims priority to U.S. Application No. 62/888,534, filed on Aug. 18, 2019, the contents of which are incorporated herein by reference in their entirety.

### BACKGROUND OF INVENTION

Difluprednate is a topical corticosteroid useful for the treatment of inflammation and pain associated with ocular surgery. It is a butyrate ester of 6α-9α-difluoro prednisolone acetate with the structure shown below.

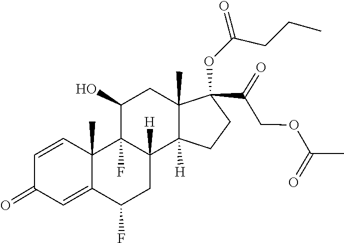

Difluprednate is practically insoluble in water. DUREZOL®, a current marketed ophthalmic formulation of difluprednate, is in an emulsion dosage form and includes 0.05% w/v difluprednate emulsified between castor oil phase and water phase. It has been used for treating inflammation and pain associated with ocular surgery and endogenous anterior uveitis when administered four times a day.

However, Durezol® emulsion formulation does not provide prolonged action which is a serious drawback. It requires to be administered four times a day, causing high rates of patient non-compliance and missing doses. Additionally, it has been reported and noted in the approved label of Durezol® that the most common adverse reactions in patients (subjects) administered with Durezol® (occurring in 5-10% of such patients) include blurred vision, eye irritation, eye pain, headache, increased intraocular pressure (IOP), iritis, limbal and conjunctival hyperemia, and punctate keratitis. Therefore, formulations of difluprednate with less or no such side effects are desirable.

In addition, U.S. Pat. No. 10,092,514 B2 discloses a difluprednate oil-in-water emulsion for treating macular edema, and US 2012/0135947 discloses an oil-in-water emulsion including difluprednate and tobramycin for topical administration. Like Durezol®, the formulations discloses in these patents require castor oil as the hydrophobic component to form emulsions. Although castor oil has been used in may ophthalmic solutions such as Restasis® and Durezol®, it may cause side effects such as itchy, redness, irritation and other uncomfortable eye issues that have also been identified with use of Durezol®. Additionally, castor oil may cause allergic reactions to some patients.

Besides oil-in-water emulsion formulations, US 2018/0311159 discloses an ophthalmic solution containing difluprednate as the sole active ingredient at a concentration of 0.02% to 0.04% in an aqueous vehicle, wherein the solution is free of oil and the solution is administered twice a day. This ophthalmic solution requires a crystal growth inhibitor to prevent the difluprednate from being precipitated or crystallized out from the aqueous solution. The crystal growth inhibitor is polyvinyl alcohol or its derivative. Polyvinyl alcohol is found in ophthalmic solutions as a lubricant to prevent irritation or to relieve dryness of the eyes. However, its use may cause temporarily blurred vision, minor burning/stinging/irritation, and even but rare serious allergic reactions.

In difluprednate formulations disclosed in prior art, either emulsion (in which castor oil is used) or crystal growth inhibitor (polyvinyl alcohol or its derivatives) was used to overcome the low solubility of difluprednate, but these additives bring highly undesirable side effects.

The present invention provides a solution to the issues discussed above that are associated with existing difluprednate formulations.

### BRIEF DESCRIPTION OF THE INVENTION

Generally speaking, the present invention provides novel difluprednate formulations based on in-situ gel technology. The novel formulations of the present invention increase drug retention time in the eye and increase the bioavailability of difluprednate (the active ingredient) in the eye. Each in-situ gel formulation provided by the present invention is an aqueous formulation and is free of oil, which has less side effects. The in-situ gel formulations of this invention can prevent difluprednate from being precipitated without using of any crystal growth inhibitor. Meanwhile, in-situ gel sustained release technology can also reduce adverse reactions such as eye irritation, eye pain and foreign body sensation in the eye. Additionally, the in-situ gel technology may further combine with suitable solubilizer/surfactant to increase the solubility and/or form nanocarriers to form smaller particles, which increase the drug permeability and drug efficacy.

The in-situ gel delivery system of the present invention prolongs the retention time of the drug in front of the cornea, which helps to improve the bioavailability of the drug in the eye. Ideally, the in-situ gel system is a low-viscosity, free-flowing liquid during storage, which allows the eye drops to be used repeatedly and easily on the eye. After administration on the conjunctival sac, it forms a semi-solid gel which adheres to the front of the eye. The viscosity should be sufficient to withstand the shear forces in the eye and prolong the retention time of the drug (difluprednate) in the front of the eye. Extended release drugs can help improve bioavailability, reduce systemic absorption, reduce the frequency of medications, and thereby improve patient compliance.

Accordingly, in one aspect, the present invention provides an aqueous in-situ gel ophthalmic formulation, comprising water, difluprednate and a biocompatible polysaccharide, wherein a gel is formed in situ at physiological temperature with instant viscosity increase upon instillation of the formulation into an eye.

Examples of a suitable biocompatible polysaccharide include deacetylated gellan gum (DGG), sodium alginate, carrageenan, hyaluronic acid, and any combination thereof. In some embodiments, the polysaccharide is DGG.

Difluprednate or the polysaccharide can be contained in the formulation at a concentration that results in most therapeutic effect and least side effects, e.g., 0.01-10.0% by weight, 0.01-5.0% by weight, 0.01-2.5% by weight, or 1% or 1.5% by weight.

The aqueous in-situ gel formulation of the present invention may further include an osmolarity adjuster, a pH adjustor, a surfactant or solubilizer, a viscosity-increasing agent, or an anti-infective agent. Each of these optional additions can have a concentration of 0.01-10.0% by weight, 0.01-5.0% by weight, 0.01-2.5% by weight, or 1% or 1.5% by weight.

Examples of a suitable osmolarity adjuster include sodium chloride, mannitol, glycerol, polyethylene glycol 400 (PEG400), boric acid, and any combination thereof. Examples of a suitable pH adjuster include sodium hydroxide, trishydroxymethylaminomethoane (Tris), hydrochloride, phosphoric acid, boric acid, and any combination thereof. Examples of a suitable surfactant or solubilizer include polyoxyethylene surfactant, polyoxypropylene surfactant, PEG 35 Caster Oil, PEG 40 Caster Oil, ethoxylated hydrogenated castor oil, Polyoxyl 40 Stearate, Soluplus, and any combination thereof. Examples of a suitable viscosity-increasing agent include polyvinyl alcohol, polyvinylpyrrolidone, methyl cellulose, hydroxyethylcellulose, carboxymethylcellulose, microcrystalline cellulose, carboxymethyl cellulose sodium, and any combination thereof.

In some embodiments of the formulation of this invention, the surfactant or solubilizer is Soluplus (a polyvinyl caprolactam-polyvinyl acetate-polyethylene glycol graft copolymer (PCL-PVAc-PEG)), which has the following formula:

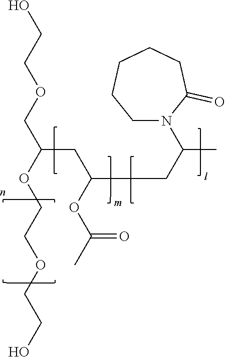

In some embodiments of the present invention, the anti-infective agent is an antibiotic or antiseptic agent. Examples of a suitable anti-infective agent include povidone-iodine (or other iodine-containing compound), netilmicin, tobramycin, doxycycline hyclate, and ciprofloxacin.

In some embodiments of the present invention, the formulation includes nanocarriers formed by the surfactant or solubilizer, with or encapsulating difluprednate, and the nanocarriers have an average particle size of 10 to 500 nm (or 10 to 250 nm, 10 to 200 nm, 10 to 150 nm, 10 to 100 nm, or 10 to 50 nm). Such nanocarriers may be micelles formed as a result of the presence of a solubilizer or surfactant which also increases the solubility of difluprednate. When the nanocarriers are formed by a surfactant or solubilizer with difluprednate, difluprednate and the surfactant or solubilizer together form the micellar membrane; whereas when the nanocarriers are formed by a surfactant or solubilizer encapsulating difluprednate, difluprednate is contained inside the membrane formed by the hydrophilic terminal of the surfactant.

The combination of in-situ gel systems (based on a particular biocompatible polysaccharide) with nanocarrier/micelle delivery systems can not only improves difluprednate's membrane transport through the nanocarrier, but also increases the permeability of difluprednate to the biofilm, improves difluprednate's stability, drug solubility, and provides targeted delivery in a sustained manner.

Another aspect of the present invention provides a method for treating or alleviating symptoms of an eye disorder in a patient (subject) in need of such treatment or alleviation. The method includes administering to the patient or subject a therapeutically effective amount of an aqueous in-situ gel ophthalmic formulation as described above. The formulation forms a gel in situ upon instillation into eyes, and releases difluprednate into eyes in a sustained manner.

Examples of such an eye disorder include inflammatory disorders or pain in the eye, particularly inflammation or pain associated with ocular surgery (during or after).

### BRIEF DESCRIPTIONS OF THE DRAWINGS

FIG. 1 shows viscosity data of Formulation 1.

FIG. 2 shows viscosity data of Formulation 2.

FIG. 3 shows viscosity data of Formulation 3.

FIG. 4 shows viscosity data of Formulation 4.

FIG. 5 shows viscosity data of Formulation 5.

FIG. 6 shows release profiles (percentages) of Formulation 3 (in-situ gel micelle solution) and Formulation 6 (emulsion solution) over time.

FIG. 7 is an illustration of micelle.

### DETAILED DESCRIPTION OF THE INVENTION

The formulation in this invention is an aqueous composition including difluprednate and a water-soluble biocompatible polysaccharide which forms a gel in situ upon instillation of the formulation onto eyes. The formulations in the invention are useful for the treatment of inflammatory disorder of the eye, such as inflammation and pain associated with ocular surgery.

Specifically, the formulations of this invention are aqueous compositions contain difluprednate as the active ingredient and a biocompatible polysaccharide as the in-situ gelling material or matrix.

As used herein, the term “in situ gel” refers to a system which is applied as a solution or suspension and is capable of undergoing rapid sol-to-gel transformation triggered by external stimulus (such as temperature, pH etc.) on instillation.

The polysaccharide contained in the formulations of this invention may include deacetylated gellan gum (DGG), Carrageenan, and sodium alginate, or a mixture of these materials. Deacetylate gellan gum may be preferred, with a concentration ranging from 0.05% to 1% (w/w).

The formulations in this invention may additionally include an osmotic pressure regulator, a pH regulator, a surfactant, a viscosity increasing agent and other pharmaceutical acceptable ingredients.

The suitable osmotic pressure regulators contained in the formulations for this invention may include sodium chloride, mannitol, glycerol, polyethylene glycol 400 (PEG400) or boric acid. The concentration of the osmotic pressure regulator may range from 0.1 to 5.0% (w/w)

The suitable pH regulators in the formulations for this invention include sodium hydroxide, trishydroxymethylaminomethoane (Tris), hydrochloride (HCl), phosphoric acid or boric acid. The final pH of the formulations may be in the range of 3.5-8.0, preferably in the range of 4.0-6.0.

The suitable surfactants contained in the formulations for this invention include polyoxyethylene surfactant, polyoxypropylene surfactant, PEG 35 Castor Oil, PEG 40 Castor Oil, Polyoxyethylene hydrogenated castor oil, Polyoxyl 40 Stearate, Soluplus or any combination thereof. The surfactant in the pharmaceutical compositions can have a concentration ranging from 0.01% to 5%.

As used herein, the term “nanocarriers” is interchangeable with “micelles” or “nanomicelles” and means aggregates (or supramolecular assemblies) of surfactant molecules dispersed in a liquid colloid.

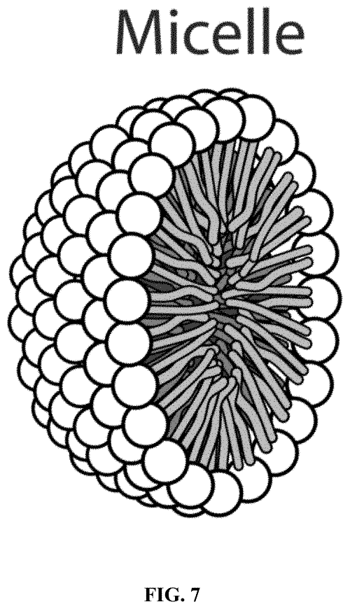

Micelles are approximately spherical in shape. Other phases, including shapes such as ellipsoids, cylinders, and bilayers, are also possible. The shape and size of a micelle are a function of the molecular geometry of its surfactant molecules and solution conditions such as surfactant concentration, temperature, pH, and ionic strength. The process of forming micelles is known as micellization and forms part of the phase behavior of many lipids according to their polymorphism. Illustrated in FIG. 7 is a spherical micelle.

The suitable viscosity-increasing agents for this invention include polyvinyl alcohol, polyvinylpyrrolidone, methyl cellulose, hydroxyethylcellulose, carboxymethylcellulose, microcrystalline cellulose, carboxymethyl cellulose sodium or any of their combinations. The concentration of the viscosity-increasing agent may range from 0.01% to 2% (w/w).

The formulations in the invention may additionally include an anti-infective agent as the second active ingredient. The anti-infective agent in the invention may be an antibiotic, an iodine-containing compound or other suitable anti-infective agent for ophthalmic formulations. The antibiotic may be netilmicin, tobramycin, doxycycline hyclate, ciprofloxacin or other suitable antibiotics. The iodine-containing compound can be an iodophor with includes iodine complexed with a solubilizing agent, such as Povidone-iodine.

The formulation in the invention may optionally include an antimicrobial preservative. Suitable antimicrobial preservatives may be added to prevent multi-dose package contamination, though the optional antibiotic agent may serve as self-preservative. Such agents may include benzalkonium chloride, thimerosal, chlorobutanol, methyl paraben, propyl paraben, phenylethyl alcohol, EDTA, sorbic acid, Onamer M, other agents known to those skilled in the art, or a combination thereof. Typically, such preservatives are employed at a level of from 0.001% to 1.0% (w/w).

The invention is further elucidated with specific examples. It is understood that these examples are only used to describe the invention but not intend to limit the scope of invention. The experimental methods with no specific conditions in the following examples, are usually prepared under conventional conditions in the literature or according to the conditions suggested by the excipient manufacturer. Unless specifically stated, all percentages, ratios, proportions or fractions in this invention are calculated by weight by weight. Unless specifically defined in this invention, all professional and scientific terms used herein have the same meaning as well-trained personnel may be familiar with. In addition, any methods and materials similar or equivalent to those recorded in this invention can be applied to this invention. The preferred embodiments and materials described herein are used only for exemplary purposes.

### Example 1: Formulation of Difluprednate In-Situ Gel Suspension

Different polysaccharides including deacetylated gellan gum (DGG), Xanthan gum, kappa-carrageenan, sodium alginate, and sodium hyaluronate were screened to select the optimum ophthalmic gel forming matrix. The formulation with Xanthan gum or sodium hyaluronate failed to show in-situ gelling ability. The viscosity of formulations with Xanthan or sodium hyaluronate did not increase after mixing with artificial tears. While the formulation with carrageenan or sodium alginate showed viscosity increase after mixing with artificial tears which demonstrated some in-situ gelling property, the viscosity after mixing with artificial tears is too low (<50 cp) and thus the in-situ gelling property for the formulation is not optimal. If using carrageenan or sodium alginate as the gel forming agent, additional ingredient such as a suitable viscosity-increasing agent is required to optimize the formulation. The formulations with DGG in general exhibited in-situ gelling ability under physiological conditions when DGG concentrations were optimized. Thus, DGG was chosen as the gel forming matrix in the formulation.

Formulation Preparation Process: Sodium chloride and mannitol was dissolved in water for injection. Gellan gum was then added slowly into the solution and heated to 60-70° C. to be fully dissolved. Then the solution was cooled to room temperature to provide Solution 1. Difluprednate was dispersed in glycerin to provide suspension 2. Suspension 2 was added into the solution 1 and mix well. The pH of the final suspension was adjusted to pH 5.5 with tromethamine. A typical formulation (Formulation 1) is showed in Table 1. The suspension is stable and the solid was not precipitated from the suspension for at least 2 months at room temperature.

TABLE 1
Formulation of Difluprednate in-situ Gel Suspension (Formulation 1)
Formulation 1

| Ingredients | Percentage(wt %) | Function |
|---|---|---|
| Difluprednate | 0.05 | Active |
| Sodium Chloride | 0.20 | Ion strength regulator |
| DGG | 0.25 | Gel former |
| Glycerol | 2.30 | Osmolality regulator |
| Sorbic Acid | 0.01 | Preservative |
| Tromethamine | q.s. to pH 5.5 | pH regulator |
| Water for Injection | q.s. | Solvent |

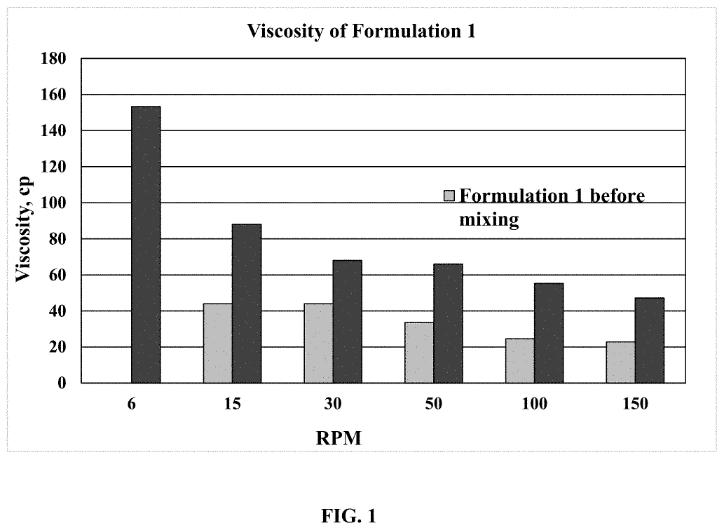

The viscosity of the sample with and without mixing with artificial tears (0.678% NaCl, 0.218% NaHCO₃, 0.0084% CaCl₂.2H₂O and 0.138% KCl in water) was tested at 33° C. The mixing ratio of sample and artificial tears is 3:7. Table 2 and FIG. 1 showed the viscosity at different shear rate. The viscosity decreased with increasing shear rate for both conditions. It was found that the viscosity significant increased after mixing with artificial teats, showing in-situ gel property.

TABLE 2
Viscosity test results of Formulation 1

Viscosity, cp

| RPM | Before Mixing | After Mixing |
|---|---|---|
| 6 | — | 153.3 |
| 15 | 44.0 | 88.0 |
| 30 | 44.0 | 68.0 |
| 50 | 33.6 | 66.0 |
| 100 | 24.6 | 55.2 |
| 150 | 22.8 | 47.2 |

The in-situ gel suspension in the example can prevent aggregation and precipitation of difluprednate. However, it did not increase the solubility and thus the permeability of the drug. To provide better solubility and bioavailability, micronized difluprednate may be required as decreasing the particle size can improve the solubility and permeability.

### Example 2: Selection of Suitable Surfactants/Solubilizers

To improve the solubility of difluprednate, different surfactants/solubilizers were investigated to discover suitable solubilizers. Different surfactants/solubilizers such as Poloxamer 188, Poloxamer 407, polysorbate 80, PEG 40 Caster Oil, PEG 60 Caster Oil, PEG 40 Hydrogenated Castor Oil, Polyoxyl 40 Stearate and Soluplus was dissolved in water with different concentrations. Difluprednate was added into the surfactant solutions with the final difluprednate concentration of 0.05%. The solubility of difluprednate was measured. Table 3 showed the solubility of difluprednate with different surfactants/solubilizer. It was found that common solubilizer such as poloxamer 188, polysorbate 80 cannot effectively increase the solubility of the difluprednate. Soluplus was surprisingly found to be the optimal solubilizer for difluprednate as the solubility of difluprednate in the formulation reached over 99% with only 0.6% Soluplus addition. Soluplus is a polyethylene glycol, polyvinyl acetate and polyvinylcaprolactame-based graft copolymer (PVAc-PVCap-PEG). It can form nanomicelles in water or other aqueous solutions, and solubilize poorly soluble difluprednate.

Besides Soluplus, Polyoxyethylene castor oil surfactants can also improve the solubility of difluprednate. The solubility of difluprednate is 99.5% with 5% Polyoxyethylene Castor Oil (EL-40) and >98% with 5% Polyoxyethylene Castor Oil (RL-40). Polyoxyethylene (60) Castor Oil and Polyoxyethylene Castor Oil (EL-35) can also increase the solubility of difluprednate to >95%, though it was found that at least 4 or 5% of such solubilized are needed to reach over 95% solubility for difluprednate. Therefore, Soluplus was a preferred solubilizer.

TABLE 3
Solubility of Difluprednate with different Surfactant/Solubilizers

| | Amount of | |
|---|---|---|
| | Surfactant/Solubilizers | |
| Surfactant/Solubilizers | (%) | Solubility % |
| Poloxamer188 | 0.1 | 3.59 |
| Poloxamer 407 | 0.2 | 2.20 |
| Polysorbate 80 | 0.05 | 1.75 |
| Hypromellose | 0.5 | 17.6 |
| Polyoxyethylene castor oil (EL-35) | 5.0 | 82.9 |
| Vitamin E Polyethylene | 0.5 | 25.3 |
| Glycol Succinate | | |
| Polyoxyethylene (40) Stearate | 7.0 | 5.1 |
| Polyoxyethylene (40) Monostearate | 0.5 | 42.0 |
| Polyoxyethylene (23) Cetyl Ether | 0.2 | 7.87 |
| | 0.45 | 15.41 |
| | 1.0 | 33.70 |
| Sucrose Fatty Acid Ester | 0.2 | 2.01 |
| | 1.0 | 8.36 |
| Glyceryl Tricaprylate | 0.2 | 0.84 |
| Benzyl Benzoate | 2 | 0.37 |
| Polyoxyethylene Castor Oil (EL) | 5.0 | 93.5 |
| Polyoxyethylene (60) Castor Oil | 5.0 | 95.3 |
| Polyoxyethylene Castor | 5.0 | >98.0 |
| Oil (EL-40) | | |
| Polyoxyethylene Castor | 5.0 | 96.3 |
| Oil (EL-35) | | |
| Polyoxyethylene | 0.53 | 9.7 |
| Hydrogenated Castor | 1.0 | 18.6 |
| Oil (RH-40) | 2.0 | 50.0 |
| | 3.0 | 73.1 |
| | 4.0 | 88.1 |
| | 5.0 | 99.5 |
| Polyoxyl 20 Cetostearyl Ether | 1.0 | 37.2 |
| | 2.0 | 74.7 |
| | 3.0 | 79.9 |
| | 4.0 | 96.9 |
| | 5.0 | 96.9 |
| Soluplus | 0.6 | 99.1 |
| | 0.9 | 101.9 |
| | 1.2 | 102.1 |
| | 1.5 | 102.1 |

### Example 3: Formulation of Difluprednate In-Situ Gel Solution with Soluplus as Solubilizer

Difluprednate in-situ gel nanomicellar solution was prepared with Soluplus as the solubilizer. The formulations were prepared with the similar method described in Example 1. Two solutions with Soluplus were obtained with the formulations showed in Table 4.

TABLE 4
Formulation of Difluprednate in-situ
Gel Solution (Formulations 2 and 3)

| | Percentage(wt %) | | |
|---|---|---|---|
| Ingredients | Formulation 2 | Formulation 3 | Functions |
| Difluprednate | 0.05 | 0.05 | Active |
| Soluplus | 0.90 | 0.90 | Solubilizer |
| Sodium Chloride | 0.20 | 0.25 | Ion strength |
| | | | regulator |
| DGG | 0.25 | 0.25 | Gel former |
| Mannitol | 2.30 | 2.30 | Osmolality |
| | | | regulator |
| Sorbic Acid | 0.01 | 0.01 | Preservative |
| Tromethamine | q.s. to pH 5.5 | q.s. to pH 5.5 | pH regulator |
| Water for Injection | q.s. | q.s. | Solvent |

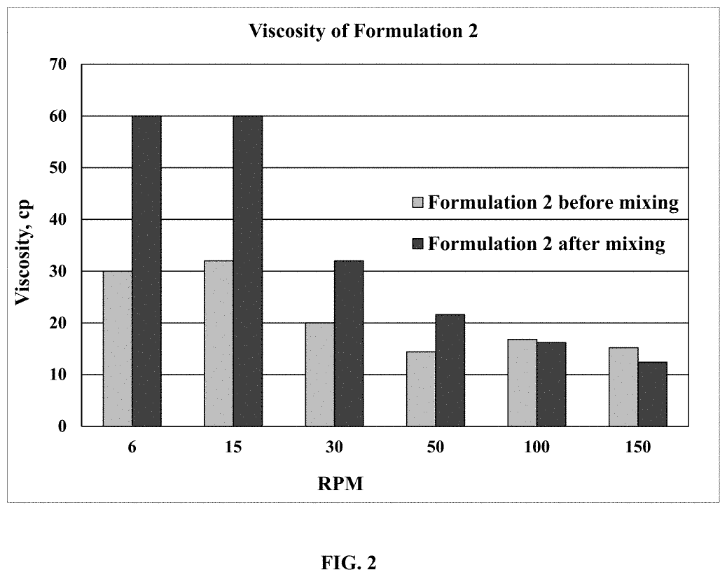

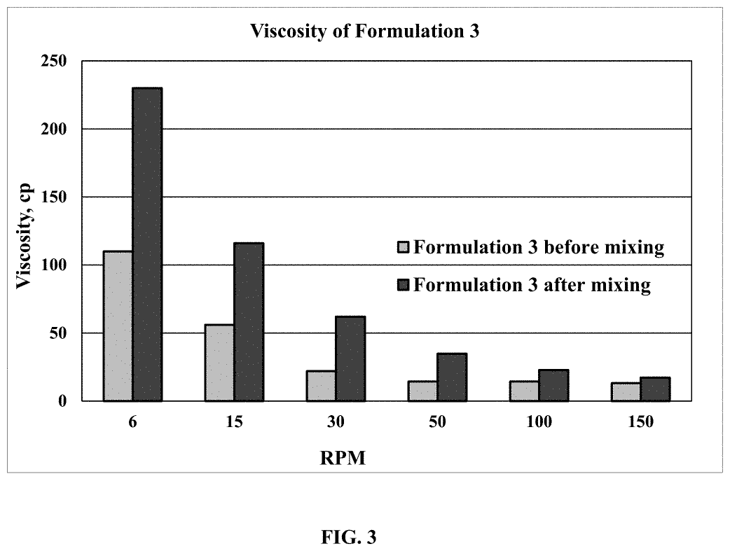

Particle size was measured and the mean particle size was 74.5 nm for Formulation 2 and 67.0 nm for Formulation 3, indicating nanomicelle was formed by addition of Soluplus. Viscosity of the two formulations was test. Table 5, FIG. 2 and FIG. 3 show the viscosity of the above two formulations tested with and without artificial tears. For Formulation 2, in-situ gel was very weak as the viscosity is much lower compared with Formulation 1. Therefore, it was found that the addition of Soluplus as a solubilizer into an in-situ gelling formulation actually weaken the gel forming property of gellan gum, resulting a lower viscosity. For formulation 3 in which the Sodium Chloride concentration increased from 0.20% to 0.25%, significant in-situ gel was formed after mixing with artificial tears. Therefore, we can optimize the ion strength of the formulation to achieve a better in-situ gel nanomicelle formulation.

TABLE 5
Viscosity test results of Formulations 2 and 3

| | Formulation 2 | | Formulation 3 | |
|---|---|---|---|---|
| | Before | After | Before | After |
| RPM | Mixing, cp | Mixing, cp | Mixing, cp | Mixing, cp |
| 6 | 30.0 | 60.0 | 110.0 | 230.0 |
| 15 | 32.0 | 60.0 | 56.0 | 116.0 |
| 30 | 20.0 | 32.0 | 22.0 | 62.0 |
| 50 | 14.4 | 21.6 | 14.4 | 34.8 |
| 100 | 16.8 | 16.2 | 14.4 | 22.8 |
| 150 | 15.2 | 12.4 | 13.2 | 17.2 |

### Example 4: Formulation of Difluprednate In Situ Gel Formulation with RH-40 as Solubilizer

Difluprednate in-situ gel formulation was prepared with Polyoxyethylene Hydrogenated Castor Oil (RH-40) as the solubilizer with the similar method described in Example 1. Two solutions were obtained with the formulation showed in Table 6. 1% RH-40 and 0.8% RH-40 was used in these formulations as the FDA IIG safety requirement for RH-40 is not more than 1%.

TABLE 6
Formulation of Difluprednate in-situ
Gel Solution (Formulations 4 and 5)

| | Percentage(wt %) | | |
|---|---|---|---|
| Ingredients | Formulation 4 | Formulation 5 | Functions |
| Difluprednate | 0.05 | 0.05 | Active |
| RH-40 | 1.00 | 0.80 | Solubilizer |
| Sodium Chloride | 0.20 | 0.16 | Ion strength |
| | | | regulator |
| DGG | 0.25 | 0.20 | Gel former |
| Mannitol | 2.30 | 2.30 | Osmolality |
| | | | regulator |
| Sorbic Acid | 0.01 | 0.01 | Preservative |
| Tromethamine | q.s. to pH 5.5 | q.s. to pH 5.5 | pH regulator |
| Water for Injection | q.s. | q.s. | Solvent |

Particle size of Formulations 4 and 5 were measured and it was found that no micelle was formed for these two formulations. Slight white suspension may be observed during storage at room temperature but no solid precipitated from the formulations. It was surprisingly found that additional of even 5% RH-40 with difluprednate did not result in micelle formation. Minor suspension may be observed after storage for 3 days or longer, thus solution stability is not as good as in-situ gel micelle solutions.

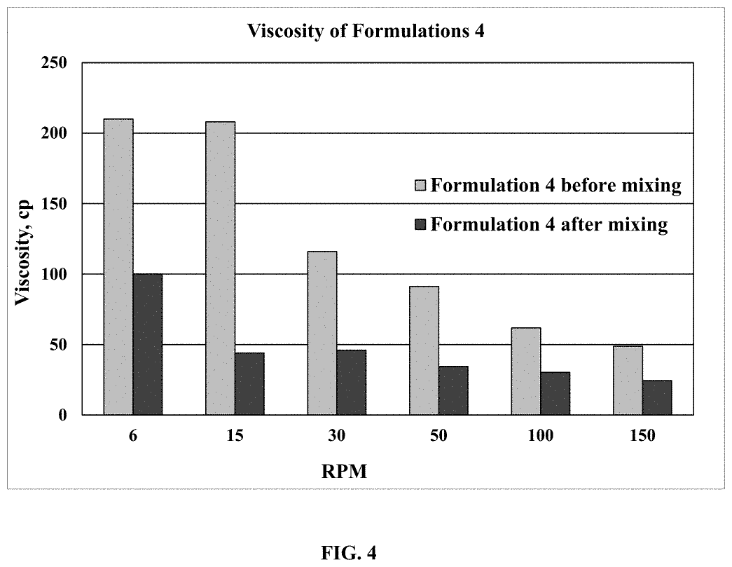

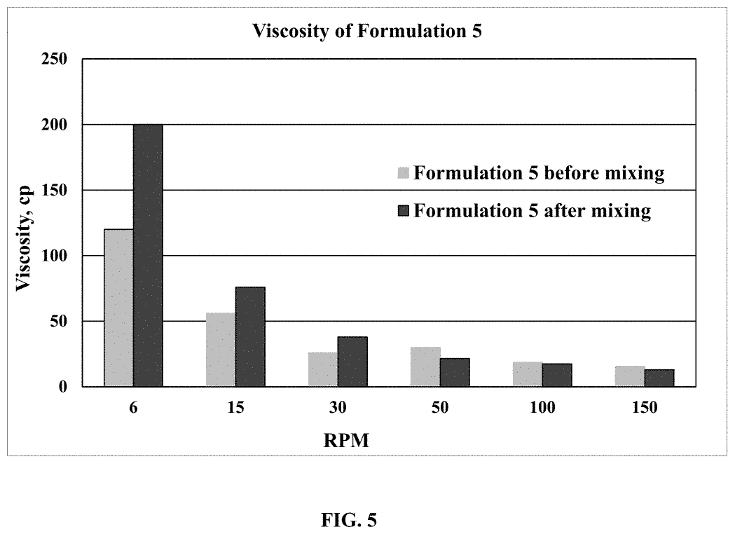

Table 7, FIG. 4 and FIG. 5 showed the viscosity of the above two formulation before and after mixing with artificial tears. It was found that gel was formed even before mixing with artificial tears for Formulation 4. Compare with Formulations 1 and 2 in which the same concentrations of Gellan Gum and Sodium Chloride were used, it was found that use of RH-40 as the solubilizer increased the gel forming and thus the gel was formed even before mixing with artificial tears. Decreasing the concentration of Gellan Gum, Sodium Chloride and RH-40 (Formulation 5) can prevent the initial gel forming and in-situ gel was formed only after mixing with artificial tears. It was also found that formulation with 5% RH-40 and 0.2% Gellan Gum, sodium chloride is not required to form suitable in-situ gel formulation. The viscosity was 100 cp at 6 RPM while the viscosity increased to 160 cp after mixing with artificial tears.

TABLE 7
Viscosity test results of Formulations 4 and 5

| | Formulation 4 | | Formulation 5 | |
|---|---|---|---|---|
| | Before | After | Before | After |
| RPM | Mixing, cp | Mixing, cp | Mixing, cp | Mixing, cp |
| 6 | 210.0 | 100.0 | 120.0 | 200.0 |
| 15 | 208.0 | 44.0 | 56.0 | 76.0 |
| 30 | 116.0 | 46.0 | 26.0 | 38.0 |
| 50 | 91.2 | 34.5 | 30.0 | 21.6 |
| 100 | 61.8 | 30.3 | 18.6 | 17.4 |
| 150 | 48.8 | 24.4 | 15.6 | 13.0 |

### Example 5: In-Vitro Dissolution Study

To evaluate the in-vitro release of the in-situ gel micelle formulation, Formulation 3 from Example 3 was selected for the dissolution study as it can form suitable in-situ gel based on the viscosity test and the solution stability is optimal due to the formation of micelle.

Difluprednate emulsion formulation (Formulation 6) was prepared as the control with the same formulation of commercial Durezol® as showed in Table 8. Briefly, Difluprednate was dissolved in Castor Oil as the oil phase. Glycerin, Polysorbate 80, Boric acid, Sodium Acetate, Sodium EDTA and Sorbic Acid were dissolved in water for injection. The pH of the water solution was adjusted to pH 5.5 as the water phase. The oil phase was added into the water phase and the mixture was homogenized with a homogenizer. The particle size of obtained solution was measure and the mean size is 123.7 nm, indicating emulsion was successfully formed. Formulation 6 was used as a control to study the extended release ability for in-situ gel solution (Formulation 3).

In-vitro release study was performed with a dissolution method. Firstly, 1 g sample (in-situ gel solution or emulsion solution) and 4 g artificial tears were placed in a 50-ml plastic tube and let it set down for 5 min to form in-situ gel for in-situ gel solution. Then 35 g PBS buffer (pH 7.4 with 0.05% SDS) was added slowly through the wall of the tube to avoid agitating the bottom solution. The 1 g solution sample from top was collected at 10 min, 20 min, 30 min and 1 hr. After solution sample was collected each time, 1 g PBS buffer was added in to keep the dissolution medium at 40 g total. The concentration of the difluprednate was measured using HPLC method. The total concentration of difluprednate for each formulation was obtained by shaking the dissolution solution and then taking 1 g sample for HPLC analysis.

TABLE 8
Formulation of Difluprednate emulsion formulation (Formulation 6)
Formulation 6

| Ingredients | Percentage(wt %) | Function |
|---|---|---|
| Difluprednate | 0.05 | Active |
| Castor Oil | 5.0 | Hydrophobic component |
| Glycerin | 2.2 | Demulcent, Osmotic agent |
| Polysorbate 80 | 4.0 | Emulsifier |
| Boric acid | 0.1 | Buffer |
| Sodium Acetate | 0.05 | Buffer |
| Sodium EDTA | 0.02 | Stabilizer |
| Sodium hydroxide | q.s. to pH 5.5 | pH regulator |
| Sorbic Acid | 0.1 | Preservative |
| Water for Injection | q.s. | Hydrophilic component |

It was observed that a gel was formed for Formulation 3 in-situ gel micelle solution when mixing with artificial tear solutions. When PBS solution was added, the gel was slowly swelled and the gel matrix gradually expanded from the bottom of the tube to the top. The in vitro dissolution study was conducted for 60 minutes and the gel maintained at the end of the study and did not completely erode. For Formulation 6, it was found that no gel was formed and the total solution inside the tube quickly became uniform.

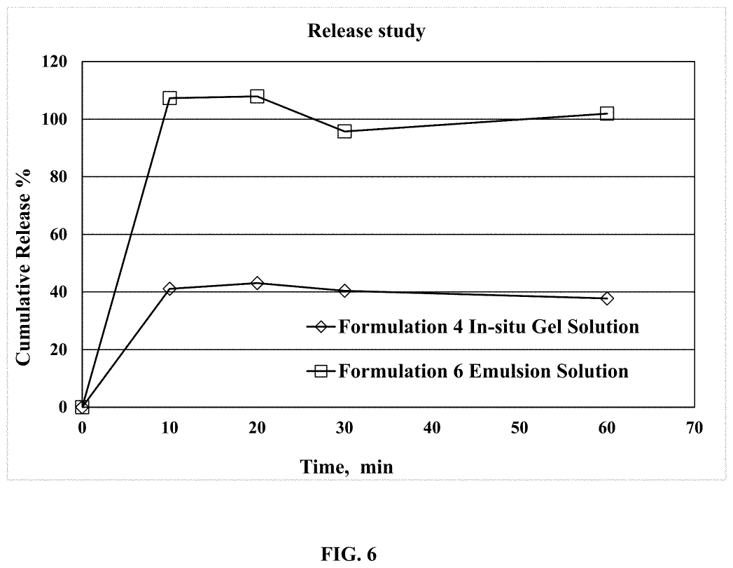

FIG. 6 shows the cumulative release percentage of difluoprednate of Formulation 3 and Formulation 6. It is surprisingly found that only 40% of the difluprednate was released after 1-hr for Formulation 3 while 100% release was achieved within 10 min for Formulation 6. In-situ gel was formed in Formulation 3 and maintained gel matrix through the study, although the gel swelled. 60% of the difluprednate was still contained inside the gel and did not release after 1-hr in the current in-vitro study. Gel was not degraded in current in-vitro study. It is expected in-vivo condition is different from the current in-vitro condition. Gel is expected to slowly degrade in-vivo and washed away by tears, and thus the loaded drug in gel is expected to slowly release in the eyes. The in-vitro release study demonstrated that the formed in-situ gel can release partial of difluprednate (40% for Formulation 3) and extend the release of the remaining difluprednate (60%). The in-situ gel is expected to be disintegrated slowly in-vivo and release the difluprednate in an extended manner.

1. An aqueous in-situ gel ophthalmic formulation, comprising water, difluprednate and a biocompatible polysaccharide, wherein a gel is formed in situ at physiological temperature with instant viscosity increase upon instillation of the formulation into an eye.

2. The aqueous in-situ gel formulation of claim 1, wherein difluprednate is contained in the formulation at a concentration of 0.01-5.0% by weight.

3. The aqueous in-situ gel formulation of claim 1, wherein the biocompatible polysaccharide comprises deacetylated gellan gum (DGG), sodium alginate, carrageenan, hyaluronic acid, or a combination thereof.

4. The aqueous in-situ gel ophthalmic formulation of claim 1, wherein the biocompatible polysaccharide is contained in the formulation at a concentration of 0.01-5% by weight.

5. The aqueous in-situ gel formulation of claim 1, wherein the biocompatible polysaccharide comprises deacetylated gellan gum (DGG).

6. The aqueous in-situ gel formulation of claim 1, further comprising an osmolarity adjuster, a pH adjustor, a surfactant or solubilizer, a viscosity-increasing agent, or an anti-infective agent.

7. The aqueous in-situ gel formulation of claim 6, wherein each of the osmolarity adjuster, the pH adjustor, the surfactant or solubilizer, the viscosity-increasing agent, or the anti-infective agent is contained in the formulation at a concentration of 0.01-5% by weight.

8. The aqueous in-situ gel formulation of claim 6, wherein the osmolarity adjuster comprises sodium chloride, mannitol, glycerol, polyethylene glycol 400 (PEG400), boric acid, or any combination thereof.

9. The aqueous in-situ gel formulation of claim 6, wherein the pH adjuster comprises sodium hydroxide, trishydroxymethylaminomethoane (Tris), hydrochloride, phosphoric acid, boric acid, or any combination thereof.

10. The aqueous in-situ gel formulation of claim 6, wherein the surfactant or solubilizer comprises polyoxyethylene surfactant, polyoxypropylene surfactant, PEG 35 Caster Oil, PEG 40 Caster Oil, Polyoxyethylene Hydrogenated Castor Oil, Polyoxyl 40 Stearate, Soluplus, or a combination thereof.

11. The aqueous in-situ gel formulation of claim 6, wherein the surfactant or solubilizer comprises Soluplus.

12. The aqueous in-situ gel formulation of claim 6, wherein the viscosity-increasing agent comprises polyvinyl alcohol, polyvinylpyrrolidone, methyl cellulose, hydroxyethylcellulose, carboxymethylcellulose, microcrystalline cellulose, carboxymethyl cellulose sodium, or any combination thereof.

13. The aqueous in-situ gel formulation of claim 6, wherein the anti-infective agent is an antibiotic or antiseptic agent.

14. The aqueous in-situ gel formulation of claim 6, wherein the anti-infective agent comprises povidone-iodine, netilmicin, tobramycin, doxycycline hyclate, or ciprofloxacin.

15. The aqueous in-situ gel ophthalmic formulation of claim 6, wherein nanocarriers are formed by the surfactant or solubilizer, with or encapsulating difluprednate, wherein difluprednate is either part of micellar membrane or encapsulated within the nanocarriers, and the nanocarriers have an average particle size of 10 to 500 nm.

16. The aqueous in-situ gel ophthalmic formulation of claim 15, wherein the nanocarriers have an average particle size of 10 to 150 nm or 10 to 50 nm.

17. A method for treating or alleviating symptoms of an eye disorder in a subject in need thereof, comprising administering to the subject a therapeutically effective amount of an aqueous in-situ gel ophthalmic formulation of claim 1.

18. The method of claim 17, wherein disorder is an inflammatory disorder or pain in an eye.

19. The method of claim 18, wherein the disorder is inflammation or pain associated with ocular surgery.
